(19) United States
(12) Patent Application Publication
(10) Pub. No.: US 2024/0316350 A1
(43) Pub. Date: Sep. 26, 2024

(54) CONFORMAL ANTENNA FOR AN IMPLANTABLE MEDICAL DEVICE

(71) Applicant: NeuroPace, Inc., Mountain View, CA (US)

(72) Inventors: Alister Hoss, Sherman Oaks, CA (US); Tygo A. Ebenhahn, San Francisco, CA (US); Natalya A. Bruner, Santa Cruz, CA (US); Stephen T. Archer, Sunnyvale, CA (US)

(21) Appl. No.: 18/124,222

(22) Filed: Mar. 21, 2023

(51) Int. Cl. A61N 1/372; A61N 1/375

(52) U.S. Cl. CPC A61N 1/37229; A61N 1/375

(57) ABSTRACT

An implantable medical device (IMD) configured for implant in a body includes an enclosure that houses communication circuitry, an antenna, a dielectric layer, and a transmission line. The enclosure includes an outer surface configured to be oriented in a direction facing the exterior of the body and to be placed adjacent tissue. The antenna includes a ground plane corresponding to the enclosure, a dielectric spacer that conforms to the outer surface of the enclosure, and a radiating element that conforms to the dielectric spacer. The dielectric layer overlays the radiating element and the dielectric spacer. The transmission line is electrically connected between the radiating element and the communication circuitry.

### TECHNICAL FIELD

The present disclosure relates generally to antennas for implantable medical devices, and more particularly, to conformal antennas associated with a surface of an enclosure or housing of an implantable medical device.

### BACKGROUND

Active implantable medical devices are known that can be configured to communicate with external components wirelessly, such as via a form of telemetry. The communication may be desirable to, for example, download information acquired by and stored on the implanted medical device to an external component, such as patient-controlled external component. Alternatively, or additionally, an external component configured as a programmer may be brought into communication with the implantable medical device to obtain data from the device or to send data to the device from the programmer, such as new programming instructions that control whatever it may be that the implantable medical device is configured to do. For example, the implantable medical device may be programmed to measure electrographic signals sensed from the patient and/or detect electrographic events whenever such events occur in the electrographic signals, or deliver a form of electrical stimulation to the patient.

A communication link between the implantable medical device and an external apparatus may be established with an intermediate device, such as wand for near-field or short-range telemetry. For example, the implantable medical device may have a telemetry coil that enables transmission and reception of signals, to or from an external apparatus, via inductive coupling. Alternatively, a communication link between the implantable medical device and an external component may be established with an antenna for a radio frequency (RF) link. Far field or long-range telemetry may obviate the need for the intermediate device (e.g., the wand) and allow the external apparatus to be further away from the implantable medical device than is the case with near-field telemetry.

### SUMMARY

An implantable medical device (IMD) configured for implant in a body includes an enclosure that houses communication circuitry, an antenna, a dielectric layer, and a transmission line. The enclosure includes an outer surface configured to be oriented in a direction facing the exterior of the body and to be placed adjacent tissue. The antenna includes a ground plane corresponding to the enclosure, a dielectric spacer that conforms to the outer surface of the enclosure, and a radiating element that conforms to the dielectric spacer. The dielectric layer overlays the radiating element and the dielectric spacer. The transmission line is electrically connected or coupled between the radiating element and the communication circuitry.

It is understood that other aspects of apparatuses and methods will become readily apparent to those skilled in the art from the following detailed description, wherein various aspects of apparatuses and methods are shown and described by way of illustration. As will be realized, these aspects may be implemented in other and different forms and its several details are capable of modification in various other respects. Accordingly, the drawings and detailed description are to be regarded as illustrative in nature and not as restrictive.

### BRIEF DESCRIPTION OF THE DRAWINGS

Various aspects of apparatuses and methods will now be presented in the detailed description by way of example, and not by way of limitation, with reference to the accompanying drawings, wherein:

FIG. 1 is perspective, schematic illustration of a neurostimulation system implanted in a patient, including a neurostimulator with a conformal antenna and a pair of leads.

FIG. 2A is an illustration of a neurostimulator with a conformal antenna and internal transmission lines between the antenna and circuitry.

FIG. 2B is a schematic cross-section of the neurostimulator taken along line 2B-2B of FIG. 2A that shows elements of a conformal antenna.

FIG. 2C is a schematic cross-section of the neurostimulator taken along line 2C-2C of FIG. 2A that shows elements of a conformal antenna.

FIG. 3A is an illustration of the conformal antenna of FIG. 2A and associated components including antenna feedthroughs.

FIGS. 3B and 3C are different perspectives of FIG. 3A with some components of FIG. 3A removed to better illustrate one of the antenna feedthroughs.

FIGS. 4A and 4B are illustrations of the conformal antenna of FIG. 3A.

FIG. 4C is an illustration of the radiating element of the conformal antenna of FIG. 3A

FIGS. 5A-5C are schematic illustrations of some possible configurations of a radiating element of a conformal antenna.

FIG. 6 is a schematic cross-section of components of a conformal antenna coupled to circuitry of the neurostimulator through an internal transmission line.

FIGS. 7A and 7B are illustrations of an internal transmission line.

FIG. 7C is a cross section of an internal transmission line.

FIG. 8A is an example graph of return loss of an internal transmission line as a function of frequency.

FIG. 8B is an example graph of insertion loss of an internal transmission line as a function of frequency.

FIG. 8C is an example graph of radiated emission or unwanted leakage out of an internal transmission line as a function of frequency.

FIG. 9A is an illustration of a neurostimulator with a conformal antenna with reference coordinates.

FIG. 9B is an example graph of radiation gain of the conformal antenna of the neurostimulator of FIG. 9A as a function of angular offset relative to different planes (XZ cut and YZ cut) when the neurostimulator is implanted in a patient's skull as shown in FIG. 1.

FIG. 9C is an example graph of radiation gain as a function of the conformal antenna of the neurostimulator of FIG. 9A as a function of frequency when the neurostimulator is implanted in a patient's skull as shown in FIG. 1.

FIGS. 10 and 11 are illustrations of neurostimulator with a conformal antenna and an external transmission line between the antenna and circuitry.

FIG. 12 is an illustration of components of a conformal antenna and an external transmission line within a channel or trench of a dielectric substrate of the antenna.

FIG. 12A is a detailed blow up of the area indicated as 12A in FIG. 12.

FIGS. 13A and 13B are illustrations of components of a conformal antenna and an external transmission lines on a dielectric substrate of the antenna and coplanar with a radiating element of the antenna.

FIGS. 14A-14C are illustrations of different configurations of conformal antenna with radiating elements at different locations on an enclosure of a neurostimulator.

FIGS. 15A and 15B are schematic illustration of a traditional patch antenna.

FIG. 16 is a schematic illustration of active electric fields generated by the patch antenna of FIGS. 15A and 15B.

FIG. 17 is a graph of return loss as a function of frequency for the patch antenna of FIGS. 15A and 15B.

FIGS. 18A and 18B are schematic illustration of a conformal antenna.

FIG. 19 is a schematic illustration of active electric fields generated by the conformal antenna of FIGS. 18A and 18B.

FIG. 20 is a graph of return loss as a function of frequency for the traditional patch antenna of FIGS. 18A and 18B.

### DETAILED DESCRIPTION

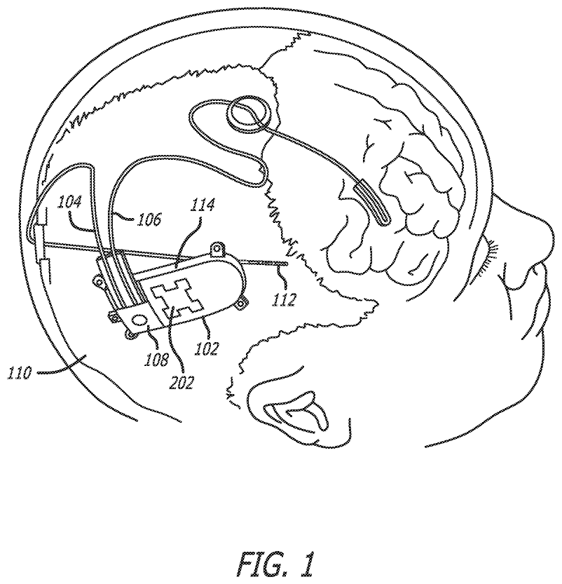

With reference to FIG. 1, an example implantable medical system in the form of a neurostimulation system is implanted in a patient. The neurostimulation system includes an implantable medical device (IMD), referred to going forward as a neurostimulator 102, and two electrode-bearing brain leads 104, 106. The neurostimulation system is configured to sense and record electrical brain activity, to detect electrographic events in the electrical brain activity, and to deliver responsive neurostimulation therapy. Responsive neurostimulation systems are described in, for example, U.S. Pat. No. 6,016,449 to Fischell, et al. for “System for Treatment of Neurological Disorders”, issued Jan. 18, 2000, U.S. Pat. No. 6,810,285 to Pless et al. for “Seizure Sensing and Detection Using an Implantable Device,” issued Oct. 24, 2004, and U.S. Pat. No. 6,690,974 to Archer et al. for “Stimulation Signal Generator for an Implantable Device” issued Feb. 10, 2004. Each of the '449, '285 and '974 patents is hereby incorporated by reference in the entirety.

The neurostimulator 102 is configured to be secured in an opening formed through the cranium 110. To this end, a tray or ferrule 114 is placed in the opening and secured to the cranium, and the neurostimulator 102 is placed in the ferrule. The neurostimulator 102 is oriented in the ferrule 114 such that a conformal antenna 202 of the neurostimulator faces outward from the interior of the cranium and is positioned to be adjacent the patient's scalp tissue.

With continued reference to FIG. 1, the neurostimulator 102 includes a lead connector 108 adapted to receive one or more of the brain leads, such as a deep brain or depth lead 104 and a cortical strip lead 106. The depth lead is implanted so that a distal end of it is situated within the patient's neural tissue, whereas the cortical strip lead is implanted under the dura mater so that a distal end of it rests on a surface of the brain. The lead connector 108 acts to physically secure the brain leads 104, 106 to the neurostimulator 102, and facilitates electrical connection to conductors in the brain leads 104, 106 coupling one or more electrodes at or near a distal end of the lead to circuitry within the neurostimulator 102.

The proximal portion of the deep brain lead 104 is generally situated on the outer surface of the cranium 110 (and under the patient's scalp), while the distal portion of the lead enters the cranium and is coupled to at least one depth electrode 112 implanted in a desired location in the patient's brain. The proximal portion of the cortical lead 106 is generally situated on the outer surface of the cranium 110 (and under the patient's scalp), while the distal portion of the lead enters the cranium. The distal portion of the cortical lead 106 includes at least one cortical electrode (not visible) implanted in a desired location on the patient's brain. Long-range (wireless) telemetry is a form of communication between implantable medical devices (IMD) and external programmers and monitors. This communication can take place over several meters or even across rooms. Previous generation of systems used near-field inductive telemetry to communicate from implanted device to external programmer. In modern and emerging systems, radio frequencies are used for long-range telemetry allowing much longer distances of communication. Along with radio frequencies comes the demand for better, high-tuned antennas to support long-range telemetry.

### IMD Communication

An international standard for implant communication is the Medical Implant Communication Service) MICS, which operates at 401-406 MHz [ETSI, 2002]. Due to the frequency of this signal and the limited power restrictions imposed by the standard, antennas used for this communication must reside outside of the protective metal housing used in most IMDs.

Most antenna designs for IMDs are placed just outside the metal housing but within rigid encapsulation, such as epoxy, to keep the antenna structure rigid and immobile. Often this is desirable since the shape and length of the antenna can have a profound effect on the performance of the antenna. The main criterion for the performance of an antenna is its efficiency in converting electrical current into electromagnetic power. The antenna is connected to the internal hermetically sealed electronics by an insulator-to-metal feedthrough (where the insulator may be formed of a material such as a ceramic or glass). Feedthrough structures bridge the hermetic barrier.

Disclosed herein is an IMD, e.g., a neurostimulator, with a conformal antenna formed in part by an enclosure of the neurostimulator, and other components that conform to an outer surface of the enclosure. In some embodiments, a radiating element of the antenna conforms to an outer surface of the enclosure and is coupled to communication circuitry inside the enclosure by an internal transmission line. In some embodiments, a radiating element of the antenna conforms to an outer surface of the enclosure and is coupled to communication circuitry inside the enclosure by an external transmission line that conforms to, and extends along, an outer surface of the enclosure.

The conformal antenna is configured to provide efficient radiation in two or more frequency bands in a particular operating environment, wherein the conformal antenna is adjacent to biological tissue. The two frequency bands of interest are the 2.4 GHz spectrum band (2400 to 2483.5 MHz) (e.g., Bluetooth) and the 5.8 GHz spectrum band (5.15 GHz to 5.85 GHZ) (e.g., WiFi). The antenna is also configured with a radiating element that radiates with a single or dual polarizations.

### Internal Transmission Line

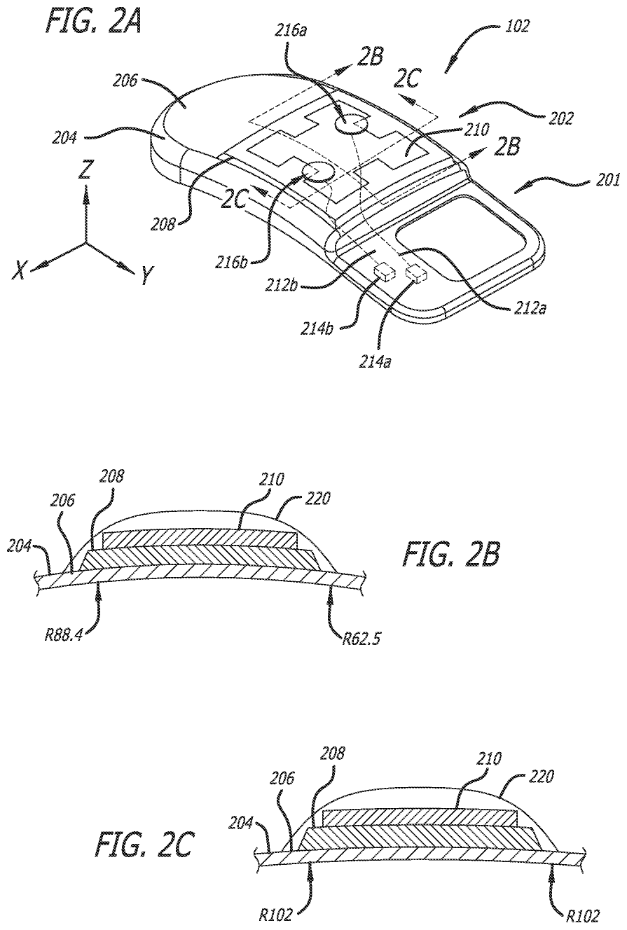

FIG. 2A is an illustration of a neurostimulator 102 with a conformal antenna 202 that includes a radiating element that conforms to an outer surface of the enclosure and is coupled to communication circuitry inside the enclosure by an internal transmission line. Some components of the neurostimulator 102 are not shown in FIG. 2A for clarity in illustrating the conformal antenna 202. For example, a connector feedthrough is not included the portion 201 of the neurostimulator 102 where the lead connector 108 couples to the neurostimulator. FIG. 2B is a schematic cross-section illustration of the neurostimulator 102 along line yz of FIG. 2A that shows elements of the conformal antenna 202. FIG. 2C is a schematic cross-section illustration of the neurostimulator 102 along line xz of FIG. 2A that shows elements of the conformal antenna 202.

With reference to FIGS. 2A, 2B, and 2C, the neurostimulator 102 includes an enclosure 204 having an outer surface 206. As shown in FIG. 1, when the neurostimulator 102 is implanted, the outer surface 206 is oriented in a direction facing the exterior of the body, and is adjacent tissue. The enclosure 204 contains electronic circuitry. For example, first communication circuitry 214a and second communication circuitry 214b may be housed within the enclosure 204. The conformal antenna 202 includes a ground plane corresponding to the enclosure 204, a dielectric spacer 208 that conforms to, and is supported by, a portion of the outer surface of the enclosure, and a radiating element 210 that conforms to, and is supported by a portion of the dielectric spacer. With reference to FIGS. 2B and 2C, a dielectric layer 220 overlays the radiating element 210, the dielectric spacer 208, and a portion of the outer surface 206 of the enclosure 204.

Dual antenna feedthroughs 216a, 216b are electrically coupled to the radiating element 210 and extend through the enclosure 204 at a location adjacent the radiating element. A respective transmissions line 212a, 212b is electrically coupled to a respective one of the antenna feedthroughs 216a, 216b to thereby provide an electrical coupling between a respective one of communication circuitry 214a, 214b and the radiating element 210.

The dual feedthrough design allows the radiating element 210 to be fed from different communications circuitry at different locations within the enclosure 204 to provide multi-band performance. For example, the first communication circuitry 214a may support Bluetooth communication, while the second communication circuitry 214b supports Wi-Fi communication.

With reference to FIGS. 2B and 2C, each of 1) the enclosure 204 wall comprising the outer surface 206 that supports the dielectric spacer 208, 2) the dielectric spacer 208, and 3) the radiating element 210 is non-planar. In other words, each of the structures (the enclosure 204 wall, the dielectric spacer 208, and the radiating element 210) has a non-linear profile that is characterized by a respective curvature. In some embodiments, these respective curvatures are substantially the same or generally corresponds to each other. Stated another way with respect to the outer surface 206 of the enclosure 204 that supports the dielectric spacer 208 and the radiating element 210, the outer surface 206 may be characterized by a non-linear profile having a first curvature and the radiating element 210 may be characterized by a non-linear profile having a second curvature that generally corresponds to the first curvature. “Generally corresponds to” as used herein means the first curvature and the second curvature are not necessarily exactly the same and that a variation between the respective curvatures is within a tolerance, e.g., less a few mils (less than 0.1 mm). For example, with reference to FIG. 2B, in one configuration the respective curvatures of the enclosure 204 wall comprising the outer surface 206, the dielectric spacer 208, and the radiating element 210 along line 2B-2B through the conformal antenna 202 has a radius of curvature R88.4 at one end of this cross-section that transitions to a radius of curvature R62.5 at the other end of the cross-section. With reference to FIG. 2C, in one configuration the respective curvatures of the enclosure 204 wall comprising the outer surface 206, the dielectric spacer 208, and the radiating element 210 along line 2C-2C through the conformal antenna 202 has a radius of curvature R102 along this cross-section.

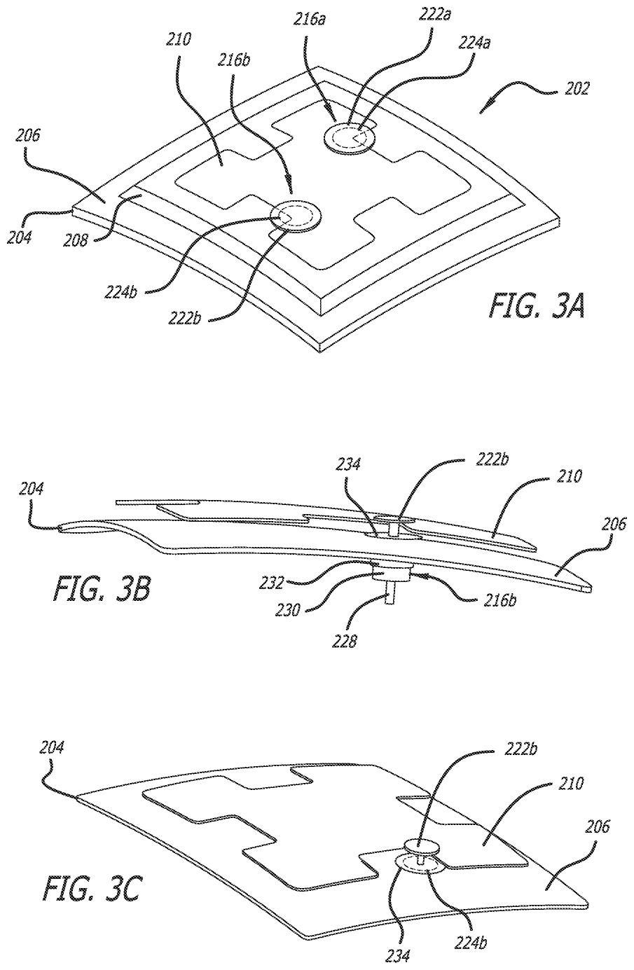

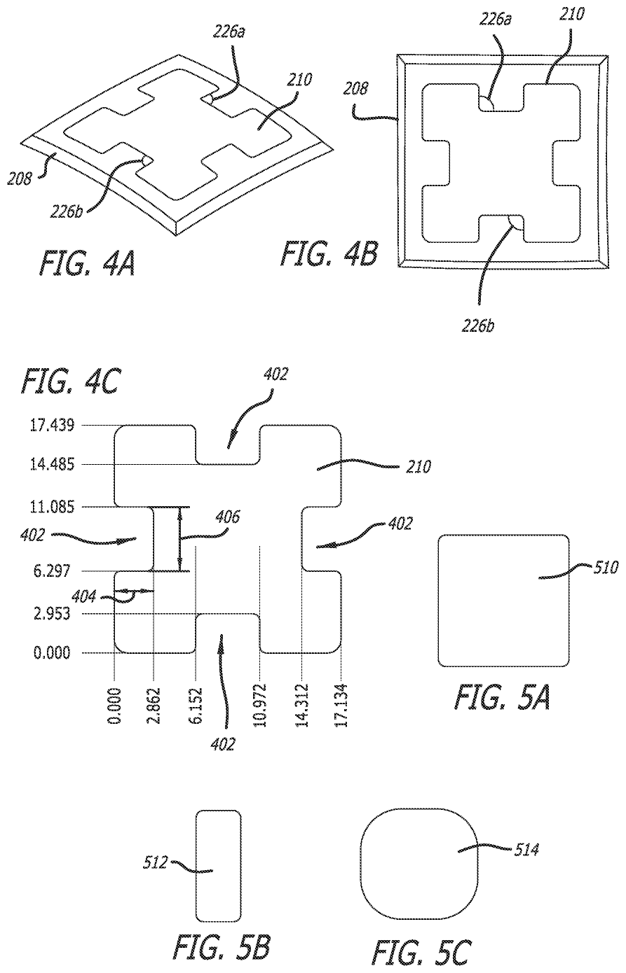

FIG. 3A is an illustration of the conformal antenna 202 and the antenna feedthroughs 216a, 216b of FIG. 2A. In FIG. 3A all components (e.g., a radiating element 210, a dielectric spacer 208, and a ground plane corresponding to an enclosure 204) of the conformal antenna 202 are shown. FIGS. 3B and 3C are different, rotated perspectives of FIG. 3A. In these figures, only one of the antenna feedthroughs is illustrated and the dielectric spacer 208 is not shown to more clearly illustrate the antenna feedthrough 216b. FIGS. 4A and 4B are illustrations of the dielectric spacer 208 and radiating element 210 of a conformal antenna 202. FIG. 4C is an illustration of a radiating element 210 of a conformal antenna 202.

With reference to FIGS. 3A-3C, the ground plane corresponding to the enclosure 204 may be formed of any electrically conductive material. For example, the enclosure 204 may be formed of any biocompatible pure conductive metal, e.g., titanium, or any biocompatible conductive alloy. In some embodiments the portion of the enclosure 204 comprising the outer surface 206 that supports the dielectric spacer 208 has a thickness in the range of 4 mil to 30 mil (˜ 0.1 mm to 0.8 mm). A pair of feedthrough holes 224a, 224b extend through the enclosure 204 wall and are configured to receive a respective antenna feedthrough 216a, 216b. As shown in FIGS. 3B and 3C, in some embodiments an antenna feedthrough 216b includes a feedthrough wire 228 that extends through a feedthrough hole 224b. The feedthrough wire 228 electrically couples to an internal transmission line 212 at one end and to a disc 222b at the other end. The disc 222b electrically couples to the surface of the radiating element 210. The electrical coupling or connecting of components may be achieved by welding, soldering or gluing (using an electrically conductive glue). One or more hermetic seals 230, 232, 234 are located on each side of the enclosure 204 wall around the feedthrough hole 224b.

With reference to FIGS. 4A and 4B, the dielectric spacer 208 may be formed of a biocompatible plastic (e.g., silicone, epoxy, PEEK) with little or no metallic/lossy add-on. In some embodiments, the dielectric spacer 208 has a thickness in the range of 4 mil to 30 mil (˜ 0.1 mm to 0.8 mm). The dielectric spacer 208 is configured to be molded or shaped (by heat) while holding its dielectric properties. The dielectric spacer 208 can be molded or shaped with the same curvature as the outer surface 206 of the enclosure 204 to thereby conform to the top surface of the enclosure.

The dielectric spacer 208 is configured to support the radiating element 210. In some embodiments, the dielectric spacer 208 has a dielectric constant (relative permittivity) that is a fraction of the dielectric constant of tissue at a specific radio frequency. For example, the dielectric constant of the dielectric spacer 208 at a specific radio frequency may be in the range of 2 to 5, while the dielectric constant of the scalp, which covers the implant and therefore loads the antenna, is in the range of 25 to 40 at 2.45 GHz. A pair of holes 226a, 226b extend through the dielectric spacer 208 and are arranged to align with the feedthrough holes 224a, 224b of the enclosure 204 and are configured to receive a respective antenna feedthroughs 216a, 216b.

The radiating element 210 is formed of a conductive material. For example, the radiating element may be formed any biocompatible pure conductive metal, e.g., titanium, or any biocompatible conductive alloy. In some embodiments the radiating element 210 has a thickness in the range of 4 mils to 30 mils (˜0.1 mm to 0.8 mm).

With reference to FIG. 4C, in one embodiment the radiating element 210 is characterized by a notched, square shape. This shape is referred to herein as a cloverleaf design. Compared to other radiating elements disclosed below and shown in FIGS. 5A, 5B, and 5C, the cloverleaf design is considered optimal as it provides excellent antenna performance while having a small footprint. In one configuration, the cloverleaf radiating element 210 is approximately 17 mm squared, and includes four notches 402, each positioned midway along the edge of the cloverleaf. Each notch 402 has a depth dimension 404 and an edge dimension 406. In the embodiment of FIG. 4C, the depth dimension is approximately 2.8 mm and the edge dimension is approximately 4.8 mm. Note, the foregoing are example dimensions that resulted from design optimizations carried out on the radiating element to achieve an acceptable size and performance.

The shape and sizes of the radiating element may vary from the cloverleaf configuration of FIG. 4C based on the desired featured designed to the antenna (e.g., the polarization, gain, initial return loss, etc.). FIG. 5A shows a radiating element 510 with a squared configuration with rounded corners. FIG. 5A shows a radiating element 510 with a squared configuration with rounded corners. FIG. 5B shows a radiating element 512 with a rectangular configuration with rounded corners. FIG. 5C shows a radiating element 514 with a squared configuration with rounded corners having a larger radius than those of FIG. 5A. The shape of the radiating element is not limited to the ones shown in the FIGS. 4C, 5A, 5B, and 5C. Any shape that can fit on the outer surface 206 of the enclosure 204 can be used.

With reference to FIGS. 2B and 2C, the dielectric layer 220 is formed of a biocompatible material. For example, the dielectric layer 220 may be a silicon-based glue-sealant. In some embodiments, the dielectric layer 220 has a thickness in the range of 4 mil to 30 mil (˜ 0.1 mm to 0.8 mm). The dielectric layer 220 is configured to be applied over the radiating element 210, the dielectric spacer 208, the portion of the outer surface 206 of the enclosure 204 that supports the radiating element 210 and the dielectric spacer 208, and the antenna feedthroughs 216a, 216b. For example, the dielectric layer 220 can be over molded onto the antenna assembly, e.g., the radiating element 210, the dielectric spacer 208, the portion of the outer surface 206 of the enclosure 204 that supports the radiating element 210 and the dielectric spacer 208, and the antenna feedthroughs 216a, 216b. Alternatively, the dielectric layer 220 can be applied as a layer that is held in place over the antenna assembly using a bonding layer such as an epoxy.

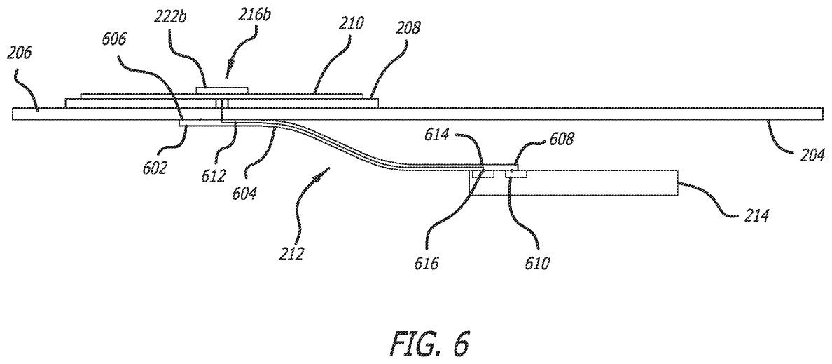

With reference to FIG. 6, the internal transmission line 212 includes a ground conductor 602 and a signal conductor 604. The ground conductor 602 has a first end 606 and a second end 608. The first end 606 is electrically connected to or coupled to the enclosure 204. To this end, the first end 606 may be welded, soldered or glued (using an electrically conductive glue) to the enclosure 204. The second end 608 is electrically connected to or coupled to a ground terminal 610 of the communication circuitry 214. To this end, the second end 608 may be welded, soldered or glued (using an electrically conductive glue) to the ground terminal 610.

The signal conductor 604 has a first end 612 and a second end 614. The first end 612 includes a pin structure that is configured to mechanically couple with the feedthrough wire 228 of the antenna feedthrough 216b and thereby electrically couple with the antenna feedthrough 216b. The second end 614 includes a pin structure that is configured to mechanically couple with a signal terminal 616 of the communication circuitry 214 and thereby electrically couple with the communications circuitry. Since the inside the neurostimulator is filled with other essential components, the internal transmission line 212 is configured to have a thin profile and mechanical flexibility.

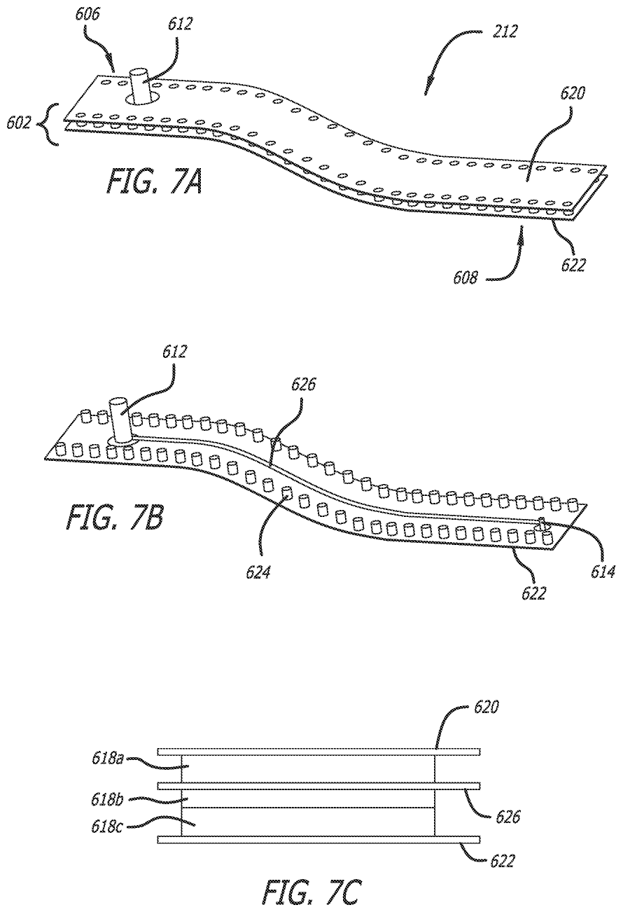

With reference to FIGS. 7A-7C, in some embodiments the internal transmission line 212 is a stacked arrangement of three flexible layers that include an upper layer 620 of electrically conductive material, a lower layer 622 of electrically conductive material, and an internal layer 626 of electrically conductive material. The electrically conductive material may be copper or another conductive metal or an electrically conductive alloy. Electrically conductive vias 624 extend between and electrically couple the upper layer 620 and the lower layer 622 to form the ground conductor 602. The upper layer 620 of the stacked internal transmission line 212 includes the first end 606 that is electrically coupled to the enclosure 204. The lower layer 622 includes the second end 608 that is electrically coupled to the ground terminal of the communication circuitry 214. The internal layer 626 forms the signal conductor 604. Dielectric layers 618a, 618b, 618c electrically isolate the ground conductor 602 and the signal conductor 604 from each other. In some embodiments, the total thickness of the stacked internal transmission line 212 is between 12-16 mils (305-405 microns).

With reference to FIG. 7C, a first dielectric layer 618a isolates the upper layer 620 of electrically conductive material from the internal layer 626 of electrically conductive material. The first dielectric layer 618a may be formed of a first flex material and has a thickness in the range of 4 mil to 10 mil (˜0.1 mm to 0.25 mm). The first flex material may be, for example, polyimide. A stacked arrangement of a second dielectric layer 618b and a third dielectric layer 618c isolates the lower layer 622 of electrically conductive material from the internal layer 626 of electrically conductive material. The second dielectric layer 618b may be formed of second flex material and has a thickness in the range of 4 mil to 10 mil (˜0.1 mm to 0.25 mm). The second flex material may be an adhesive material that functions to adhere the first dielectric layer 618a with the third dielectric layer 618c. The internal layer 626 of electrically conductive material is thus embedded in dielectric material. The second flex material may be, for example, an epoxy or an acrylic based adhesive. The third dielectric layer 618c may be formed of a third flex material and has a thickness in the range of 4 mil to 10 mil (˜0.1 mm to 0.25 mm). The third flex material 618c may be the same material as the first dielectric layer 618a. The third flex material 618c may be, for example, polyimide.

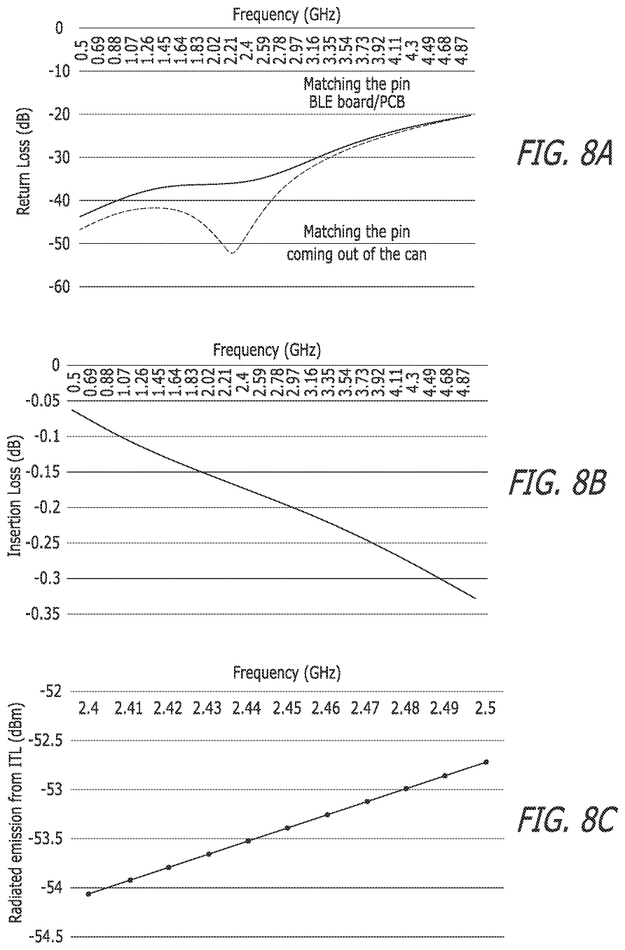

FIG. 8A is an example graph of return loss of the stacked internal transmission line 212 as a function of frequency. The return loss as a function of frequency of the internal transmission line may vary based on the design of the internal transmission line. FIG. 8B is an example graph of insertion loss of the stacked internal transmission line 212 as a function of frequency. The insertion loss as a function of frequency of the internal transmission line may vary based on the design of the internal transmission line. FIG. 8C is an example graph of radiated emission or unwanted leakage out of the stacked internal transmission line 212 as a function of frequency under simulation. The radiated emission as a function of frequency of the internal transmission line may vary based on the design of the internal transmission line.

Overall, the stacked internal transmission line 212 shows a particularly good performance in a wide frequency range of 500 MHz up to 5 GHz. As shown in FIG. 8A, the return loss of the stacked internal transmission line 212 is better than 20 dB, which is equivalent to less than 1% reflection of the input power at the connection pins of the transmission line. As shown in FIG. 8B, the insertion loss of the stacked internal transmission line 212 is better than −0.3 dB. This means that less than 7% of power is lost in the internal transmission line 212. As shown in FIG. 8C, the radiated emission out of the stacked internal transmission line 212 when the input power to the one side of the transmission line is only 0 dBm (1 mW). The radiated emission is less if −52 dBm (i.e., only 0.0000063 of the input power is emitted).

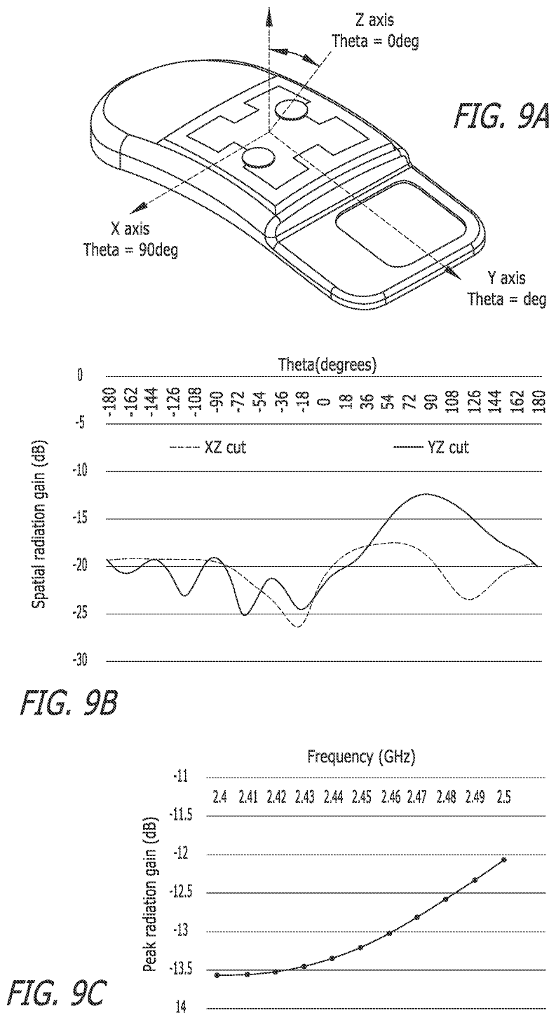

With reference to FIG. 9A, RF performance of the conformal antenna was tested under simulated implant conditions resembling those shown in FIG. 1. More specifically, the outer surface 206 of the neurostimulator 102 was simulated inside a phantom model representing a patient's head, with the conformal antenna 202 adjacent tissue under the scalp.

The peak radiation gain of the antenna was measured under these simulated conditions. Radiation gain shows the percentage/portion of the input power (i.e. from the communication circuitry) that radiates in all directions. All the environmental losses are included in this number. FIG. 9B is an example graph of radiation gain of the conformal antenna of the neurostimulator of FIG. 9A as a function of angular offset relative to different planes (XZ cut and YZ cut), when the neurostimulator is implanted in a patient's skull as shown in FIG. 1. With reference to FIG. 9B, results of the simulated test show the conformal antenna 202 has a peak radiation gain of about −13 dB (i.e., about 5%). The conformal antenna 202 being buried under the skin shows an initial return loss of ˜ 3 dB. The return loss may be improved by the use of a matching network between the communication circuitry 214 and the conformal antenna 202.

The initial return loss of the antenna was also measured under these simulated conditions. Return loss (i.e., matching) gives the percentage of the input power that gets transferred to the antenna. As an example, a return loss of 10 dB is equivalent to 10% reflection of the power back to the communication circuitry. FIG. 9C is an example graph of radiation gain as a function of the conformal antenna of the neurostimulator of FIG. 9A as a function of frequency, when the neurostimulator is implanted in a patient's skull as shown in FIG. 1. With reference to FIG. 9C, results of the simulated test show the peak gain of the conformal antenna 202 varies by about 0.5 dB (i.e., about 10%) within the entire desired frequency band (i.e. Bluetooth). The variation is well within an acceptable range.

### External Transmission Line

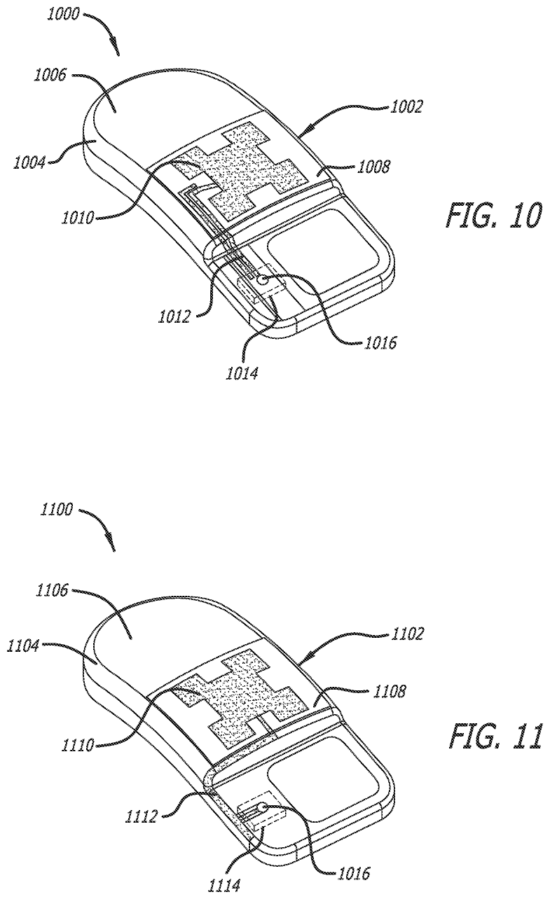

FIGS. 10 and 11 are illustrations of neurostimulators 1000, 1100 with a conformal antenna that includes a radiating element that conforms to an outer surface of the enclosure and is coupled to communication circuitry inside the enclosure by an external transmission line that conforms to, and extends along, an outer surface of the enclosure.

With reference to FIGS. 10 and 11, the neurostimulator 1000, 1100 includes an enclosure 1004, 1104 having an outer surface 1006, 1106. The enclosure 1004, 1104 contains electronic circuitry. For example, communication circuitry 1014, 1114 may be housed within the enclosure 1004, 1104. The conformal antenna 1002, 1102 includes a ground plane corresponding to the enclosure 1004, 1104, a dielectric spacer 1008, 1108 that conforms to, and is supported by, a portion of the outer surface of the enclosure, and a radiating element 1010, 1110 that conforms to, and is supported by a portion of the dielectric spacer. An external transmission line 1012, 1112 conforms to, and is supported by a portion of the dielectric spacer that extends along the outer surface 1006, 1106 of the enclosure 1004, 1104 to a location remote from the radiating element 1010, 1110. In some embodiments, the external transmission line 1012, 1112 is an electrical trace. A dielectric layer (not shown) overlays the external transmission line 1012, 1112, the radiating element 1010, 1110, the dielectric spacer 1008, 1108, and a portion of the outer surface 1006, 1106 of the enclosure 1004, 1104. An antenna feedthrough 1016, 1116 extends through the enclosure 1004, 1104 wall at the location remote from the radiating element 1010, 1110. The external transmission line 1012, 1112 electrically coupled to the antenna feedthrough 1016, 1116 to thereby couple the radiating element 1010, 1110 to the communication circuitry 1014, 1114.

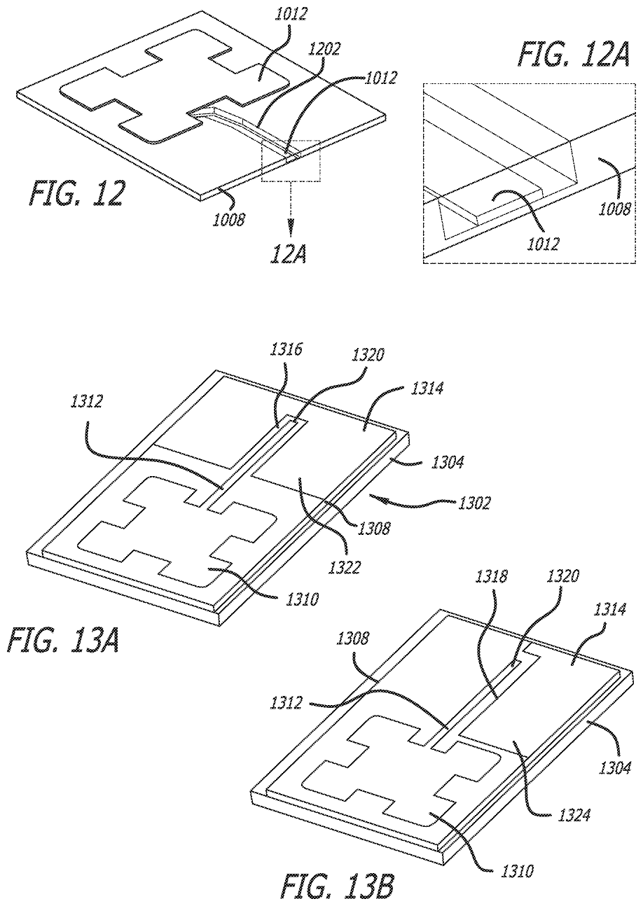

With reference to FIGS. 10, 12, and 12A, in some embodiments the external transmission line 1012 extends within a trench or channel 1202 formed within the dielectric spacer 1008. The channel 1202 extends from a location on the dielectric spacer 1008 beneath the radiating element 1010 (where the external transmission line 1012 couples to the radiating element 1010) to the location remote from the radiating element 1010 (where the external transmission line 1012 couples to the antenna feedthrough 1016). A filler 1204 encapsulates the external transmission line 1012 within the channel. The filler 1204 may be a plastic-based an insulator that functions to hold the external transmission line 1012 in place. In this embodiment, because the external transmission line 1012 lies in a channel 1202 in a plane different from the plane in which the radiating element 1010 lies, the external transmission line 1012 is not co-planar with the radiating element 1010. The channel 1202 allows for the control of the impedance of the external transmission line 1012 so that more of the signals from the communication circuitry reach to the radiating element 1010.

With reference to FIGS. 13A and 13B, in some embodiments a conformal antenna 1302 includes a rectangular structure 1314a, 1314b that is coplanar with the radiating element 1310 and functions as an extension of the ground plane corresponding to the enclosure 1304. The rectangular structure 1314a, 1314b is supported by and conforms to a portion of the dielectric spacer 1308 that is spaced apart from the portion of the dielectric spacer that supports the radiating element 1310. Thus, the rectangular structure 1314a, 1314b is co-planar with the radiating element 1310. The rectangular structure 1314a, 1314b electrically couples to the ground plane corresponding to the enclosure 1304 through a feed-through (not visible) underneath the rectangular structure.

The external transmission line 1312 extends from the radiating element 1310 to a region adjacent to and spaced apart from the rectangular structure 1314a, 1314b. The terminal end 1320 of the external transmission line 1312 electrically couples to communication circuitry (not shown) through a feed-through (not visible) underneath the external transmission line. With reference to FIG. 13A, in some embodiments, the rectangular structure 1314a spans a distance 1322 of the dielectric spacer 1308 greater than the radiating element 1310. The rectangular structure 1314a has a slot 1316 that exposes a portion of the dielectric spacer 1308 and the external transmission line 1312 extends from the radiating element 1310 into and along a length of the slot while maintaining separation from the rectangular structure 1314a. With reference to FIG. 13B, in some embodiments, the rectangular structure 1314b spans a distance 1324 of the dielectric spacer 1308 less than the radiating element 1310. The rectangular structure 1314b has a notch 1326 and the external transmission line 1312 extends from the radiating element 1310 along a length of the notch while maintaining separation from the rectangular structure 1314b. These rectangular structure 1314a, 1314b allow for the control of the impedance of the external transmission line 1312 so that more of the signals from the communication circuitry reach to the radiating element 1310.

In the configurations of FIGS. 13A and 13B, the dielectric spacer 1308 is of uniform thickness and the radiating element 1310, external transmission line 1312, and rectangular structure 1314a, 13a4b are metal printed onto the dielectric spacer 1308. The resulting structure is then secured to the enclosure 304.

### Location of Conformal Antenna

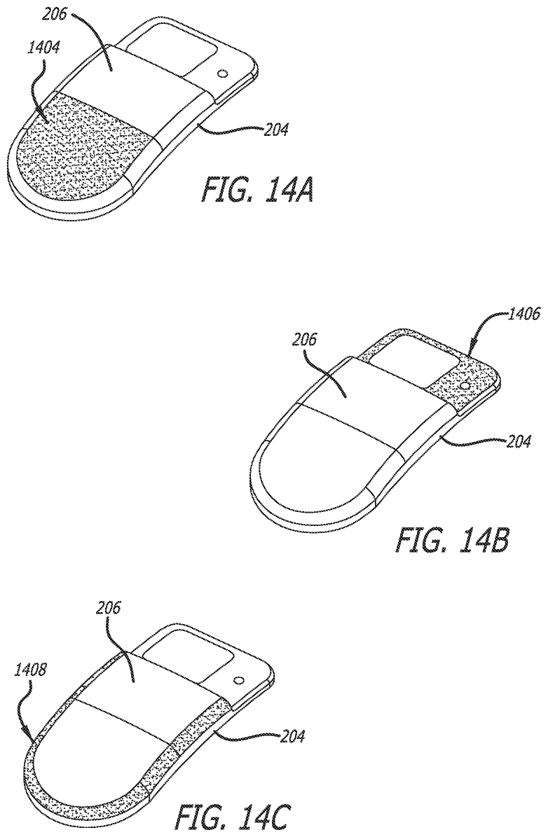

While the conformal antenna 202 primarily described throughout this disclosure has a radiating element 210 on a particular segment, e.g., a middle segment, of the outer surface 206 of the enclosure 204, a conformal antenna may have a radiating element located on other segments of the outer surface of the enclosure. For example, with reference to FIG. 14A a conformal antenna may have a radiating element on a first end segment 1404 of the outer surface 206 of the enclosure 204. With reference to FIG. 14B, a conformal antenna may have a radiating element on a second end segment 1406 of the outer surface 206 of the enclosure 204. With reference to FIG. 14C, a conformal antenna may have a radiating element on a perimeter segment 1408 of the outer surface 206 of the enclosure 204.

Assembly of Neurostimulator with Conformal Antenna

Following is an example process of assembling a neurostimulator with a conformal antenna of the type shown in FIG. 2A.

A top subassembly of the neurostimulator is assembled. To this end, a top half of an enclosure 204 is obtained. The top half of the enclosure 204 has an outer surface 206 with curved, non-linear cross-section profiles and installed antenna feedthrough 216a, 216b. A radiating element 210 having curved, non-linear cross-section profiles and a surface shape generally conforming to the contour of the outer surface 206 of enclosure 204 is obtained. A dielectric spacer 208 having curved, non-linear cross-section profiles and a surface shape generally conforming to the contour of the outer surface 206 of the enclosure 204, and with and feedthrough holes 226a, 226b that match the locations of the antenna feedthroughs 216a, 216b is obtained.

The radiating element 210 is adhered to the dielectric spacer 208. The radiating element 210 and dielectric spacer 208 are adhered to the outer surface 206 of the enclosure 204 such that the holes 226a, 226b align with the antenna feedthroughs 216a, 216b. Electrical connections are made between the feedthrough wires 228 and the radiating element 210 using the disc 222a, 222b. A dielectric layer 220 is adhered to the outer surface 206 of the enclosure to cover the assembled radiating element 210 and dielectric spacer 208. A first internal transmission line 212a is electrically connected at one end to a feedthrough wire 228 of a first antenna feedthrough 216a. A second internal transmission line 212b is electrically connected at one end to a feedthrough wire 228 of a second antenna feedthrough 216b.

A bottom subassembly of a neurostimulator is obtained. The bottom subassembly includes a bottom half of the enclosure and holds various electronics, including a first communication circuitry 214a and a second communication circuitry 214b. The first internal transmission line 212a of the top subassembly is electrically connected at its other end to the first communication circuitry 214a. The second internal transmission line 212b is electrically connected at its other end the second communication circuitry 214b. Other connections between the top and bottom subassemblies are made and the top and bottom portions of the enclosure 204 are secured together to form the neurostimulator.

### Comparison to Traditional Patch Antenna

Compared to a traditional antenna, the conformal antenna disclosed herein has some unique features. The conformal antenna is implanted in the skull while a thick layer of skin is placed over the antenna (i.e., the antenna is being loaded by the skin). The radiating element and the ground plane of the conformal antenna are both non-planar and have a similar curvature. The conformal antenna (and external transmission line if presented) is covered by a silicon-based sealant/glue so that bodily fluid and/or blood do not contact the radiating element (and external transmission line if presented), and do not penetrate the enclosure via the feedthrough beneath the radiating element to contact an internal transmission line (if present).

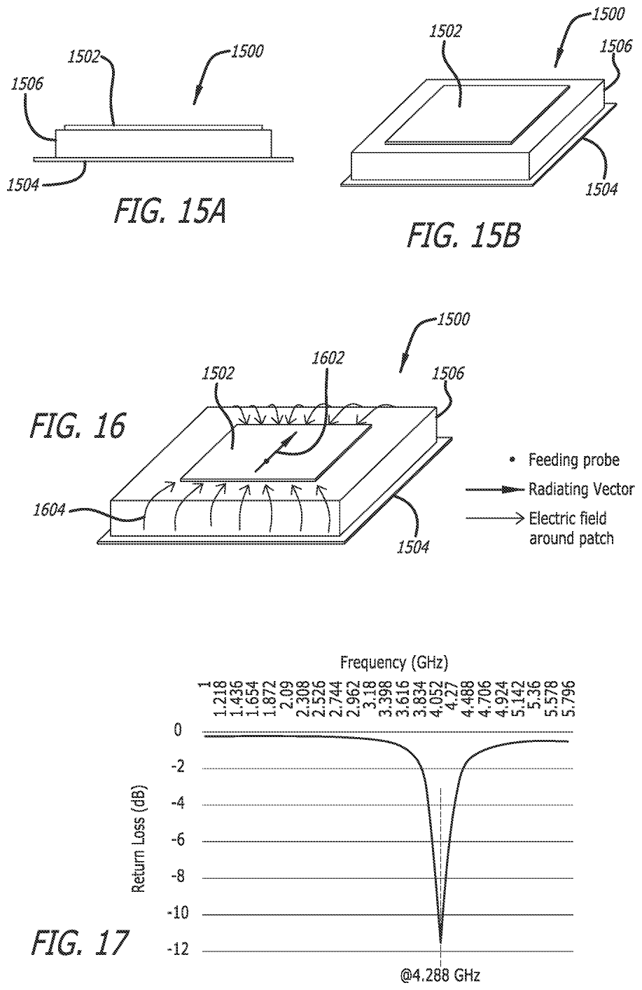

FIGS. 15A and 15B show a traditional patch antenna 1500 having two metallic layers 1502, 1504 with a substrate 1506 separating two layers. Patch antennas are usually a half-wavelength by a half-wavelength shape. The wavelength is defined as the effective wavelength of the antenna on the designated substrate as the wavelength at the center frequency in air divided by the effective permittivity of the substrate (for example, the effective wavelength of @2.45 GHz for a substrate with permittivity of 2, is about 85 mm, so an ideal patch antenna would be about 40 mm by 40 mm).

With reference to FIG. 16, in a traditional patch antenna 1500, the active electric field 1604 generating the radiating vector 1602 is mainly confined by the substrate 1506. The active electric field 1604 on the sides of the patch antenna 1500 form a constructive radiating vector 1602. Since the substrate 1506 contains the majority of the active electric field 1604, the dielectric properties of the substrate play an important role in determining the resonance frequency of the patch antenna 1500.

With reference to FIG. 17, it can be seen that the traditional patch antenna provides a peak return loss of 12 dB at 4.288 GHz under simulation in air. However, at a desired Bluetooth frequency of 2.45 GHZ, the return loss is only 0.1 dB. In order for antenna to be useable in a system, the initial return loss at the desired frequency should be at least 2 dB or better. Thus, the traditional patch antenna is unsuitable for 2.45 GHz.

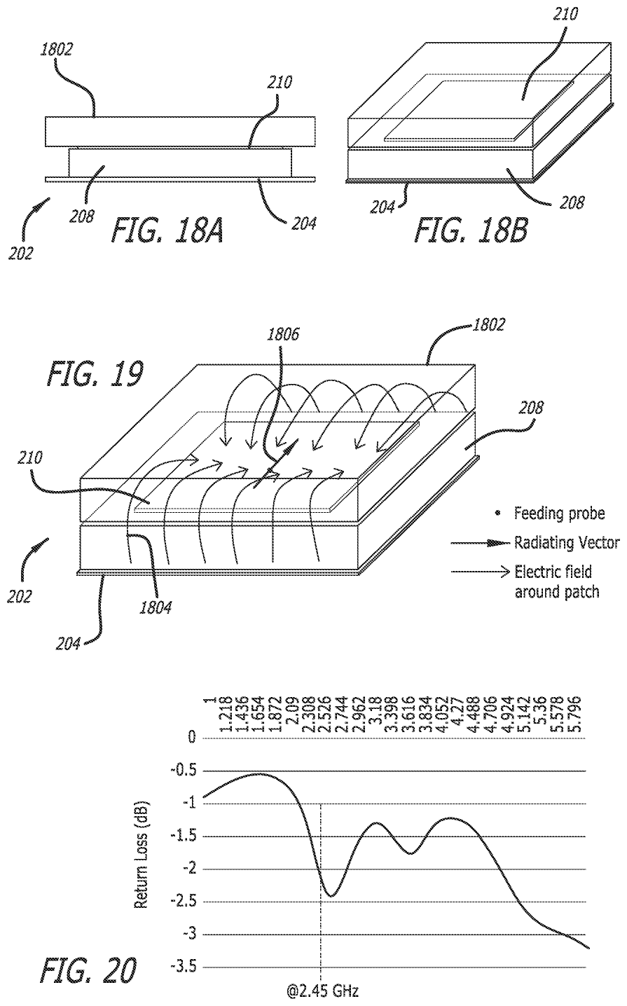

With reference to FIGS. 18A and 18B, the conformal antenna 202 disclosed herein is designed and optimized while placed in a particular operating environment (implanted under skin), therefore, the operating concept behind the conformal antenna 202 is significantly different than that of a traditional patch antenna operating in air.

The skin 1802 can be modeled as a lossy dielectric. The dielectric constant of the skin 1802 at a desired frequency (e.g., 2.45 GHZ) is over 20 while the dielectric spacer 208 of the conformal antenna 202 has a relative directivity of about 2-3. As a result, the layer of the skin 1802 is the dominant material characterizing the performance of the conformal antenna 202. Similarly, the skin 1802 has an estimated conductivity of 1 S/m while this number essentially is close to zero for regular RF substrates.

FIG. 19 shows the simplified electric field distribution of the conformal antenna 202 in a desired operating environment (implanted under skin) 1802. Since the conformal antenna 202 is loaded with skin 1802, which has a much higher dielectric constant than air and the dielectric spacer 208, the active electric field 1804 generating the radiating vector 1806 is surrounds and covers the radiating element 210. This is distinct from a patch antenna of FIG. 16, where the active electric field 1604 is confined on two edges along the radiating element 1502.

FIG. 20 shows the return loss of a conformal antenna 202 when a layer of skin covers the antenna. The initial return loss of this conformal antenna 202 at the desired frequency (e.g., 2.45 GHZ) is more than 2 dB.

The various aspects of this disclosure are provided to enable one of ordinary skill in the art to practice the present invention. Various modifications to exemplary embodiments presented throughout this disclosure will be readily apparent to those skilled in the art, and the concepts disclosed herein may be extended to other magnetic storage devices. Thus, the claims are not intended to be limited to the various aspects of this disclosure, but are to be accorded the full scope consistent with the language of the claims. All structural and functional equivalents to the various components of the exemplary embodiments described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.”

What is claimed is:

1. An implantable medical device (IMD) configured for implant in a body, the IMD comprising:
an enclosure that houses communication circuitry, and comprising an outer surface configured to be oriented in a direction facing the exterior of the body and to be placed adjacent tissue;
an antenna including:
a ground plane corresponding to the enclosure;
a dielectric spacer that conforms to the outer surface of the enclosure; and
a radiating element that conforms to the dielectric spacer;
a dielectric layer that overlays the radiating element and the dielectric spacer; and
a transmission line electrically coupled between the radiating element and the communication circuitry.

2. The IMD of claim 1, wherein the ground plane corresponding to the enclosure is non-planar.

3. The IMD of claim 1, wherein the radiating element is non-planar.

4. The IMD of claim 1, wherein the outer surface of the enclosure that supports the dielectric spacer is characterized by a non-linear profile having a first curvature and the radiating element is characterized by a non-linear profile having a second curvature that generally corresponds to the first curvature.

5. The IMD of claim 1, wherein the radiating element is characterized by a notched, rectangular shape.

6. The IMD of claim 1, wherein the radiating element is formed of a conductive material and has a thickness in a range of a 4 mils to 30 mils (˜0.1 mm up to 0.8 mm).

7. The IMD of claim 1, wherein the enclosure is formed of a conductive material and the portion of the enclosure beneath the dielectric spacer has a thickness in a range of a 4 mils to 30 mils (˜0.1 mm up to 0.8 mm).

8. The IMD of claim 1, wherein the dielectric spacer has a dielectric constant at a specific radio frequency that is a fraction the dielectric constant of tissue at the specific radio frequency.

9. The IMD of claim 8, wherein the dielectric constant of the dielectric spacer is in a range of 2 to 5.

10. The IMD of claim 1, wherein the dielectric spacer is formed of a biocompatible plastic and has a thickness in a range of a 4 mils to 30 mils (˜ 0.1 mm up to 0.8 mm).

11. The IMD of claim 1, wherein the dielectric layer is formed of a biocompatible material and has a thickness in a range of a 4 mils to 30 mils (˜ 0.1 mm up to 0.8 mm).

12. The IMD of claim 1, wherein the antenna operates in a frequency band in a range of at least one of 2.4 GHz to 2.5 GHz and from 5.15 GHz to 5.85 GHz.

13. The IMD of claim 1, wherein the antenna has a peak radiation gain in a range of −10 dB to −13 dB when implanted under skin.

14. The IMD of claim 1, further comprising second communication circuitry and a second transmission line, wherein the second transmission line is electrically coupled between the radiating element and the second communication circuitry.

15. The IMD of claim 13, wherein the antenna operates in a first frequency band in a range of 2.4 GHz to 2.5 GHZ, and in a second frequency band in a range of 5.15 GHz to 5.85 GHz.

16. The IMD of claim 1, wherein the transmission line is within the enclosure.

17. The IMD of claim 16, further comprising at least one antenna feedthrough electrically coupled to the radiating element, and that extends through the enclosure at a location beneath the radiating element, wherein the transmission line is electrically coupled to the radiating element through the at least one antenna feedthrough.

18. The IMD of claim 17, wherein the transmission line comprises:
a ground conductor having a first end electrically coupled to the enclosure, and a second end electrically coupled to a ground terminal of the communication circuitry; and
a signal conductor having a first end electrically coupled to the at least one antenna feedthrough, and a second end electrically coupled to a signal terminal of the communication circuitry.

19. The IMD of claim 18, wherein:
the transmission line further comprises a dielectric that isolates the ground conductor from the signal conductor; and
the ground conductor comprises:
a flexible upper layer of electrically conductive material, wherein the flexible upper layer includes the first end that is electrically coupled to the enclosure; and
a flexible lower layer of electrically conductive material, wherein the flexible upper layer includes the second end that is electrically coupled to the ground terminal of the communication circuitry; and
a plurality of electrically conductive vias electrically coupled between the flexible upper layer and the flexible lower layer,
wherein the dielectric is between the flexible upper layer and the flexible lower layer and the plurality of electrically conductive vias extend through the dielectric structure.

20. The IMD of claim 19, wherein the signal conductor is between the flexible upper layer and the flexible lower layer.

21. The IMD of claim 19, wherein a thickness of the transmission line is between 12-16 mils (305-405 microns).

22. The IMD of claim 1, wherein the transmission line is outside the enclosure.

23. The IMD of claim 22, further comprising at least one antenna feedthrough electrically coupled to the communication circuitry, and that extends through the enclosure at a location remote from the radiating element, wherein the transmission line is electrically coupled to the communication circuitry through the at least one antenna feedthrough.

24. The IMD of claim 22, wherein the dielectric spacer, the transmission line, and the dielectric layer extend together along the outer surface of the enclosure to a location remote from the radiating element.

25. The IMD of claim 22, wherein the transmission line extends within a channel formed within the dielectric spacer, and further comprising a filler that encapsulates the transmission line within the channel.

26. The IMD of claim 22, further comprising a rectangular structure that is:
co-planar with and spaced apart from the radiating element,
conformed to a portion of the dielectric spacer, and
electrically coupled to the ground plane through a feedthrough.

27. The IMD of claim 26, wherein the transmission line extends from the radiating element to a region adjacent to and spaced apart from the rectangular structure.

28. The IMD of claim 27, wherein the rectangular structure has a slot that exposes a portion of the dielectric spacer, and the transmission line is spaced apart from the rectangular structure and extends into and along a length of the slot.

29. The IMD of claim 27, wherein the rectangular structure has an edge, and the transmission line is spaced apart from the edge and extends along a length of the edge.
